(19) United States
(12) Patent Application Publication
(10) Pub. No.: US 2026/0130759 A1
(43) Pub. Date: May 14, 2026

(54) REPLACEMENT HEART VALVE ASSEMBLY WITH FABRIC MESH

(71) Applicant: BOSTON SCIENTIFIC SCIMED, INC., MAPLE GROVE, MN (US)

(72) Inventor: Michael G Hayes, Galway (IE)

(73) Assignee: BOSTON SCIENTIFIC SCIMED, INC., MAPLE GROVE, MN (US)

(21) Appl. No.: 19/385,849

(22) Filed: Nov. 11, 2025

Related U.S. Application Data
(60) Provisional application No. 63/718,881, filed on Nov. 11, 2024.

(51) Int. Cl. A61F 2/24

(52) U.S. Cl. CPC A61F 2/2418; A61F 2/2415; A61F 2210/0076; A61F 2230/0058

(57) ABSTRACT

A replacement heart valve assembly includes a stent frame including a plurality of struts defining a lattice structure with a plurality of strut junctions, an inner skirt coupled to an inner surface of the stent frame, an outer skirt coupled to an outer surface of the stent frame, and a fabric mesh wrapped around a portion of the stent frame, where the fabric mesh is positioned to reduce leak channels around the strut junctions.

### CROSS-REFERENCE TO RELATED APPLICATIONS

This application claims the benefit of priority of U.S. Provisional Application No. 63/718,881 filed November 11, 2024, the entire disclosure of which is hereby incorporated by reference.

### TECHNICAL FIELD

The present disclosure pertains to medical devices, systems, and methods for manufacturing and/or using medical devices and/or systems. More particularly, the present disclosure pertains to a replacement heart valve assembly.

### BACKGROUND

A wide variety of intracorporeal medical devices have been developed for medical use, for example, intravascular use. Some of these devices include guidewires, catheters, replacement heart valves, medical device systems, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices.

In an example, a replacement heart valve assembly may include a stent frame with a plurality of struts defining a lattice structure with a plurality of strut junctions, an inner skirt coupled to an inner surface of the stent frame, an outer skirt coupled to an outer surface of the stent frame, and a fabric mesh wrapped around a portion of the stent frame, where the fabric mesh is positioned to reduce leak channels around the strut junctions.

Alternatively or additionally to the example above, in another example, the fabric mesh may have a height between 1.5 mm and 2.5 mm.

Alternatively or additionally to any of the examples above, in another example, the fabric mesh may have a thickness between 30-50 μm.

Alternatively or additionally to any of the examples above, in another example, the fabric mesh may be positioned between the inner skirt and the stent frame.

Alternatively or additionally to any of the examples above, in another example, the fabric mesh may be configured to embed into the inner skirt and fold around the strut junctions.

Alternatively or additionally to any of the examples above, in another example, the fabric mesh may comprise folds and pleats formed to fill and follow a profile of the stent frame in leak channel areas.

Alternatively or additionally to any of the examples above, in another example, the fabric mesh may be configured to promote trapping of red blood cells in small porous areas of the fabric mesh.

Alternatively or additionally to any of the examples above, in another example, the fabric mesh may include a first layer extending circumferentially around the inner surface of the stent frame, covering a row of X-shaped strut junctions and positioned between the inner skirt and the stent frame.

Alternatively or additionally to any of the examples above, in another example, the assembly may further include a second layer of fabric mesh extending circumferentially around the outer surface of the stent frame, covering the row of X-shaped strut junctions and positioned between the stent frame and the outer skirt.

Alternatively or additionally to any of the examples above, in another example, the inner skirt and the outer skirt may be attached to the fabric mesh with stitches extending through the inner skirt, the fabric mesh, and the outer skirt and around the stent frame at the X-shaped strut junctions.

Alternatively or additionally to any of the examples above, in another example, the stitches may be arranged in two circumferential rows spaced apart axially.

Alternatively or additionally to any of the examples above, in another example, the fabric mesh may be made of a material selected from polyethylene terephthalate (PET), polyamide (PA), polypropylene (PP) and polyetheretherketone (PEEK).

Alternatively or additionally to any of the examples above, in another example, the stent frame may define a plurality of circumferentially adjacent X-shaped strut junctions that each includes first and second upper arms and first and second lower arms, wherein the fabric mesh may extend between the circumferentially adjacent X-shaped strut junctions and at each X-shaped strut junction, the fabric mesh may extend in an inverted U-shape over the first and second upper arms.

Alternatively or additionally to any of the examples above, in another example, the stent frame may define a plurality of circumferentially adjacent X-shaped strut junctions that each includes first and second upper arms and first and second lower arms, wherein the fabric mesh may extend in a figure-eight around the first and second upper arms and the first and second lower arms.

Alternatively or additionally to any of the examples above, in another example, the stent frame may define a plurality of circumferentially adjacent X-shaped strut junctions that each includes first and second upper arms and first and second lower arms, wherein a portion of the fabric mesh may extend around the outer surface of the stent frame, the fabric mesh including a plurality of T-shaped sections positioned such that a vertical bar of each T-shaped section may be positioned over one of the X-shaped strut junctions, and a horizontal part of each T-shaped section may define first and second arms each wrapping around one of the first and second upper arms of the X-shaped strut junction.

In an example, a replacement heart valve assembly may include a stent frame with a plurality of struts joined at a plurality of strut junctions, an inner skirt coupled to an inner surface of the stent frame, an outer skirt coupled to an outer surface of the stent frame, and at least one piece of fabric mesh wrapped around at least the plurality of strut junctions, wherein the fabric mesh may be positioned between the inner skirt and the stent frame, between the outer skirt and the stent frame, or between the stent frame and both of the inner skirt and the outer skirt.

In an example, a method of making a replacement heart valve assembly may include providing a stent frame with a plurality of struts connected to one another at strut junctions, attaching valve leaflets to the stent frame, wrapping a fabric mesh around the stent frame and positioning the fabric mesh to reduce leak channels around strut junctions, attaching an inner skirt to an inner surface of the stent frame with the fabric mesh positioned between the inner skirt and the stent frame, and attaching an outer skirt to an outer surface of the stent frame.

Alternatively or additionally to the example above, in another example, wrapping the fabric mesh around the stent frame may include forming folds and pleats in the fabric mesh to fill and follow a profile of the stent frame in leak channel areas.

Alternatively or additionally to any of the examples above, in another example, the fabric mesh may be wrapped around the stent frame and at strut junctions the fabric mesh may be wrapped in an inverted U shape across the top of each strut junction.

Alternatively or additionally to any of the examples above, in another example, the method may further include forming the fabric mesh into a T shape at each strut junction, wherein a vertical part of the T shape may cover a front of the stent frame at the strut junction, and a horizontal part of the T shape may be folded down to cover an inner part of the strut junction between the stent frame and the inner skirt.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates a prior art replacement heart stent valve;

FIG. 2 illustrates an example fabric mesh on a stent frame with inner and outer skirts;

FIGS. 3A, 3B, and 3C. are cross-sectional views of example positions of fabric mesh on a stent frame with inner and outer skirts;

FIG. 4 illustrates an example fabric mesh with folds on a stent frame with inner and outer skirts;

FIG. 5 illustrates an example method of wrapping a fabric mesh on a stent frame;

FIG. 6 illustrates another example method of wrapping a fabric mesh on a stent frame; and

FIG. 7 illustrates a further example method of wrapping a fabric mesh on a stent frame.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate example embodiments of the disclosure but not limit the disclosure. Those skilled in the art will recognize that the various elements described and/or shown may be arranged in various combinations and configurations without departing from the scope of the disclosure. However, in the interest of clarity and ease of understanding, while every feature and/or element may not be shown in each drawing, the feature(s) and/or element(s) may be understood to be present regardless, unless otherwise specified .

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “ and/or ” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “ proximal ”, “ distal ”, “advance”, “withdraw”, variants thereof, and the like, may be generally considered with respect to the positioning, direction, and/or operation of various elements relative to a user/operator/manipulator of the device, wherein “ proximal ” and “withdraw” indicate or refer to closer to or toward the user and “ distal ” and “advance” indicate or refer to farther from or away from the user. In some instances, the terms “ proximal ” and “ distal ” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “ outer extent ” may be understood to mean a maximum outer dimension, “ radial extent ” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “ minimum extent ” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “ an embodiment ”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “ second ” element, a “third” element, etc. or may be omitted entirely, and/or a different feature may be referred to as the “first” element. The meaning and/or designation in each instance will be apparent to the skilled practitioner.

Diseases and/or medical conditions that impact the cardiovascular system are prevalent throughout the world. Some mammalian hearts (e.g., human, etc.) include four heart valves: a tricuspid valve, a pulmonary valve, an aortic valve, and a mitral valve. Some relatively common medical conditions may include or be the result of inefficiency, ineffectiveness, or complete failure of one or more of the valves within the heart. For example, failure of the aortic valve or the mitral valve can lead to a serious health condition and/or death if not properly addressed. Treatment of defective heart valves poses challenges in that the treatment often requires the repair or outright replacement of the defective heart valve.

Disclosed herein is an apparatus, system, and/or method that may be used in a portion of the cardiovascular system to diagnose, treat, and/or repair the system. In some embodiments, the apparatus, system, and/or method disclosed herein may be used before and/or during a procedure to diagnose, treat, and/or repair a defective heart valve (e.g., the aortic valve, the mitral valve, etc.). In addition, a replacement heart valve implant may be delivered percutaneously and thus may be much less invasive to the patient. The apparatus, system, and/or method disclosed herein may also provide other desirable features and/or benefits as described below.

It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the leaflet”, “the strut”, or other features may be equally referred to all instances and quantities beyond one of said feature unless clearly stated to the contrary. As such, it will be understood that the following discussion may apply equally to any and/or all of the components for which there are more than one within the replacement heart valve implant and/or the apparatus unless explicitly stated to the contrary.

Additionally, it should be noted that in any given figure, some features may not be shown, or may be shown schematically, for clarity and/or simplicity. Additional details regarding some components and/or method steps may be illustrated in other figures in greater detail. The systems, devices, and/or methods disclosed herein may provide a number of desirable features and benefits as described in more detail below. For the purpose of this disclosure, the discussion below is directed toward the treatment of a native aortic valve and will be so described in the interest of brevity. This, however, is not intended to be limiting as the skilled person will recognize that the following discussion may also apply to a mitral valve or another heart valve with no or minimal changes to the structure and/or scope of the disclosure. Similarly, the medical devices disclosed herein may have applications and uses in other portions of a patient’s anatomy, such as but not limited to, arteries, veins, and/or other body lumens.

Transcatheter aortic valve implantation (TAVI) is a minimally invasive procedure used to treat aortic valve stenosis. TAVI systems are engineered to provide optimal hemodynamic performance and durability in the challenging environment of the aortic root, where they must withstand the constant pressure and flow of blood while maintaining proper valve function. When the replacement valve does not seal completely around the native aortic valve, leakage may occur in the form of blood flowing around the outside of the valve. In particular, leakage may occur in patients with high eccentricity in the native aortic roots.

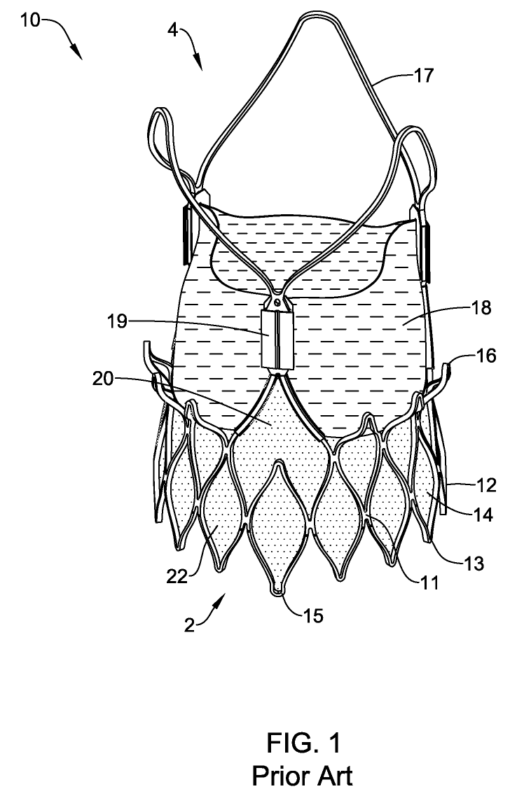

TAVI systems typically consist of an expandable frame and a valve assembly. FIG. 1 illustrates a prior art replacement heart valve implant 10 allowing one-way flow therethrough from an inflow end 2 to an outflow end 4. The replacement heart valve implant 10 includes a stent frame 12 defining a central lumen. The stent frame 12 includes a plurality of struts 13 defining a lattice structure disposed and/or extending around a central longitudinal axis. The plurality of struts 13 defines a plurality of interstices 14 (e.g., openings) between adjacent frame struts and/or through the stent frame 12. The stent frame is designed with multiple X-shaped strut junctions 11 to allow for compression during delivery and expansion once in place. The stent frame 12 defines a plurality of lower crowns 15 at the inflow end 2 of the lattice structure, a plurality of upper crowns 16 proximate an outflow end of the lattice structure, and a plurality of stabilization arches 17 extending downstream from the outflow end of the lattice structure. The plurality of stabilization arches 17 extends downstream of and/or away from the upper crowns 16 in a direction opposite the lower crowns 15.

The replacement heart valve implant 10 includes a plurality of valve leaflets 18 disposed within a central lumen of the stent frame 12. The plurality of valve leaflets 18 are coupled, secured, and/or fixedly attached to the stent frame 12. The stent frame 12 includes a plurality of commissures 19 disposed at a base of the plurality of stabilization arches 17, joining circumferentially adjacent to the stabilization arches 17. The replacement heart valve implant 10 and/or the stent frame 12 may include an inner skirt 20 attached to the inner surface of the stent frame 12, and an outer skirt 22 attached to the outer surface of the stent frame 12. The inner skirt 20, typically made of bovine or porcine pericardial tissue, may serve to channel blood within a conduit space of the stent frame 12, and obstruct leakage of blood through the plurality of interstices 14 of the stent frame 12. The outer skirt 22 may serve to provide a seal surface outside the stent frame 12 for sealing with surrounding tissue, and to obstruct leakage at the interface with surrounding tissue. The outer skirt 22 is substantially transparent, showing the underlying stent frame 12.

Previous approaches to reducing paravalvular leakage have included various skirt structures, such as standard cylindrical skirts, as shown in FIG. 1, and those with multiple ribs (not shown). However, these structures often face challenges in preventing leakage around the stent frame, particularly at the strut junctions 11, and maintaining a low profile during delivery. The present invention overcomes these limitations with a fabric mesh that provides additional material at the strut junctions 11, which reduces leakage around the stent frame while maintaining a low profile during delivery.

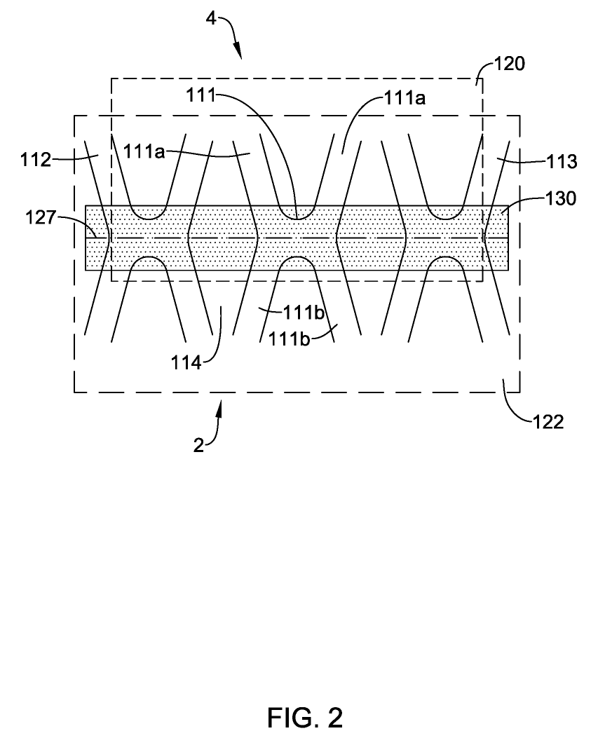

It should be appreciated that the replacement heart valve implant can be any type of heart valve (e.g., a mitral valve, an aortic valve, etc.). FIG. 2 illustrates a portion of a replacement heart valve assembly. The portions shown are similar in form and function as those shown in FIG. 1. A stent frame 112 may include a plurality of struts 113 defining a lattice structure disposed and/or extending around a central longitudinal axis. The plurality of struts 113 defines a plurality of interstices 114 between adjacent struts 113 and/or through the stent frame 112. The stent frame is designed with a plurality of X-shaped strut junctions 111 to allow for compression during delivery and expansion once in place. The plurality of X-shaped strut junctions 111 may be arranged in at least one circumferential row extending around the stent frame 112. In some embodiments, the stent frames 112 may have a single row of X-shaped strut junctions 111 while other stent frames 112 may have two or more rows of X-shaped strut junctions 111, with each row positioned axially of a previous row and the X-shaped strut junctions 111 in adjacent rows being circumferentially offset from one another. Each X-shaped strut junction 111 may include first and second upper arms 111a and first and second lower arms 111b, as shown in FIG. 2.

An inner skirt 120 may be coupled to an inner surface of the stent frame 112, between the stent frame 112 and the valve leaflets, and an outer skirt 122 may be coupled to an outer surface of the stent frame 112. The outer skirt 122 is shown transparent to allow the underlying structures to be viewed. In some embodiments, the inner skirt 120 and/or the outer skirt 122 may seal one of, some of, a plurality of, or all of the plurality of interstices 114 formed in the stent frame. In at least some embodiments, sealing the interstices may be considered to prevent fluid from flowing through the interstices from inside of the stent frame 112 to outside of the stent frame 112. In some embodiments, the inner skirt 120 and/or the outer skirt 122 may be attached to the stent frame 112 and/or the plurality of struts 113 using one or more methods including but not limited to tying with sutures or filaments, adhesive bonding, melt bonding, embedding or over molding, welding, etc.

In some embodiments, at least one piece of a fabric mesh 130 may be wrapped around a portion of the stent frame 112 and positioned around at least the strut junctions 111 to prevent fluid, such as blood, from flowing around the stent frame 112 in an upstream direction and causing paravalvular leakage. The fabric mesh 130 may ensure that fluid flows through the replacement heart valve implant only in the downstream direction from inflow end 2 to outflow end 4, and does not flow around the stent frame 112, causing regurgitation. In some embodiments, the fabric mesh 130 may be configured to embed into the inner skirt 120 and fold around the strut junctions 111, such as in the inverted U-shaped and figure-eight orientations described below. In other embodiments, the fabric mesh 130 may extend across the interstices 114 between the strut junctions 111.

The fabric mesh 130 may be configured to promote trapping of red blood cells in small porous areas of the mesh, which may reduce leaks between the stent frame 112 and the inner and outer skirts 120, 122. The fabric mesh 130 may improve sealing around the stent frame 112 by equalizing sealing ability around the stent frame 112 in the interstices 114 where there are no struts 113. The fabric mesh 130 may reduce leaks by targeting leak jets around the stent frame 112. Minimizing leaks ensures active sealing by maximizing pressure between the inner skirt 120 and the outer skirt 122 and therefore maximizing inflation on the inside of the inner and outer skirts 120, 122 during diastole so the valve self-seals to the annulus wall. The fabric mesh 130 helps to maximize pressure by reducing leak jets around the stent frame 112. Leak jets reduce inter skirt pressures and therefore active sealing. Leak jets may increase hemolysis (breakdown of red blood cells) due to high shear caused by leak jets. Filling the inner and outer skirts 120, 122 with blood at high pressure during diastole may help emptying the inner and outer skirts 120, 122 when the valve opens, producing a flush effect to empty at lower pressures. Filling and emptying the inner and outer skirts 120, 122 may be more effective during the systole cycle with reduced leak conditions as the fabric mesh 130 promotes an active sealing mechanism. The fabric mesh 130 may help reduce stagnancy and hemolysis by reducing the potential for leak channels. The fabric mesh 130 may be made of a variety of biocompatible mesh filter materials such as, but not limited to, polyethylene terephthalate (PET), polyamide (PA), polypropylene (PP) and polyetheretherketone (PEEK). The materials may be formed as medical grade interwoven meltblown fibers. The fabric mesh 130 may include a first layer extending circumferentially around the inner surface of the stent frame 112, covering a row of X-shaped strut junctions111 and positioned between the inner skirt 120 and the stent frame 112. The fabric mesh 130 may be positioned such that the bottom (inflow) end of the inner skirt 120 is even with the bottom edge of the fabric mesh 130. The fabric mesh 130 may be sewn to the stent frame 112 and the inner skirt 120 with at least one row of horizontal suture stitches 127 extending circumferentially around the stent frame 112. See FIG. 2. When a single row of stitches 127 is used, the stitches may be centered over the strut junctions 111. When two rows of stitches 127 are used, the two rows of stitches 127 may be positioned at top and bottom edges of the fabric mesh 130. The inner skirt 120 and the outer skirt 122 may be attached to the fabric mesh 130 with stitches extending through the inner skirt 120, the fabric mesh 130, and the outer skirt 122 and around the stent frame 112 at the X-shaped strut junctions 111. In some embodiments, two rows of stitches 127 may be axially spaced apart from one another such that the stitches in one row are positioned circumferentially offset from the stitches in the other row of stitches 127. The row(s) of stitches 127 may be continuous, extending around the entire circumference of the stent frame 112.

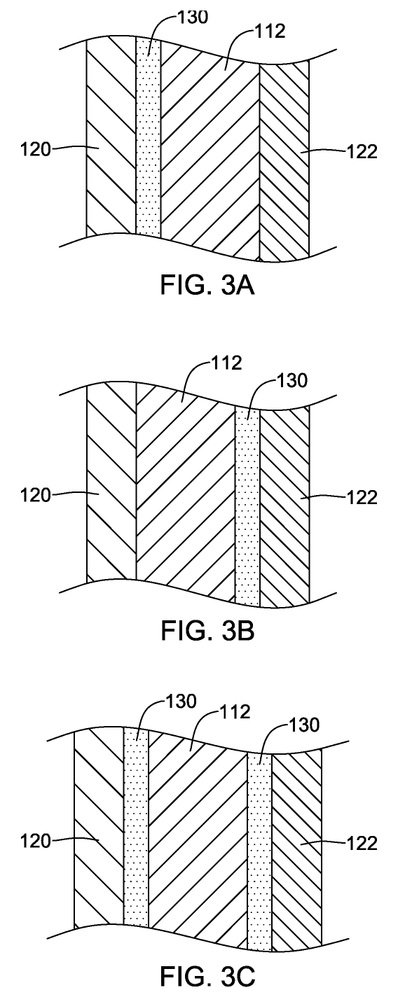

In some embodiments, the replacement heart valve assembly may include a second layer of fabric mesh 130 extending circumferentially around the outer surface of the stent frame 112, covering the row of X-shaped strut junctions 111 and positioned between the stent frame 112 and the outer skirt 122. The fabric mesh 130 may be positioned between the stent frame 112 and the inner skirt 120, as shown in FIG. 3A, between the stent frame 112 and the outer skirt 122, as shown in FIG. 3B, or two pieces of fabric mesh 130 may be used, with one between the stent frame 112 and the inner skirt 120 and one between the stent frame 112 and the outer skirt 122, as shown in FIG. 3C.

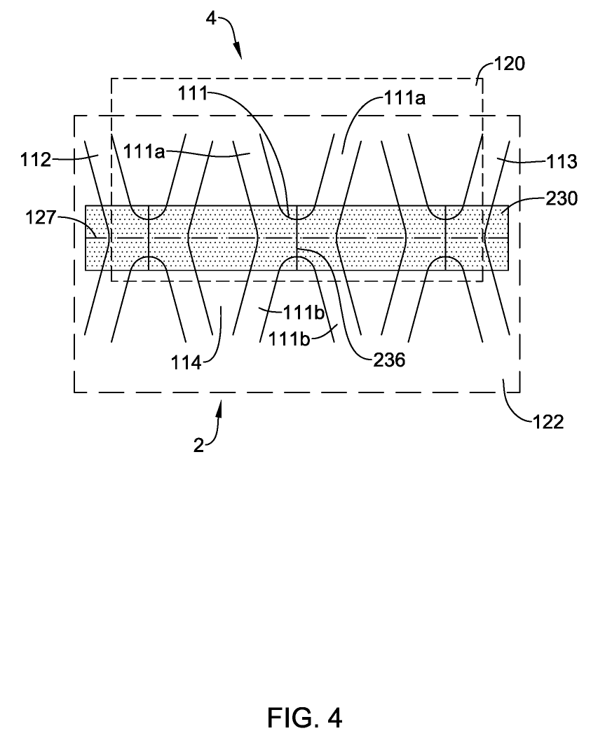

In another embodiment, a fabric mesh 230 may include folds and/or pleats 236 formed therein to fill and follow a profile of the stent frame 112 in leak channel areas such as the strut junctions 111. As shown in FIG. 4, the fabric mesh 230 has a plurality of vertical folds or pleats 236 positioned over each strut junction 111. Alternatively, the folds and/or pleats 236 may be oriented at an angle such as an angle matching the angle of the struts 113 forming the strut junctions 111. The fabric mesh 230 may be positioned between the stent frame 112 and the outer skirt 122, as shown, or the fabric mesh 230 may be positioned between the stent frame 112 and the inner skirt 120.

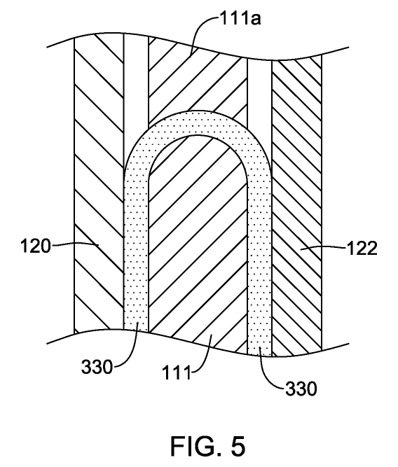

In some embodiments, a portion of the fabric mesh 330 may extend between circumferentially adjacent X-shaped strut junctions 111, and at each X-shaped strut junction 111, the fabric mesh 330 may extend in an inverted U-shape over the first and second upper arms 111a, as shown in the cross-sectional view in FIG. 5. In some embodiments, the portion of the fabric mesh 330 extending between circumferentially adjacent X-shaped strut junctions 111 may be positioned between the stent frame 112 and the inner skirt 120, and in other embodiments, the portion of fabric mesh 330 may be positioned between the stent frame 112 and the outer skirt 122.

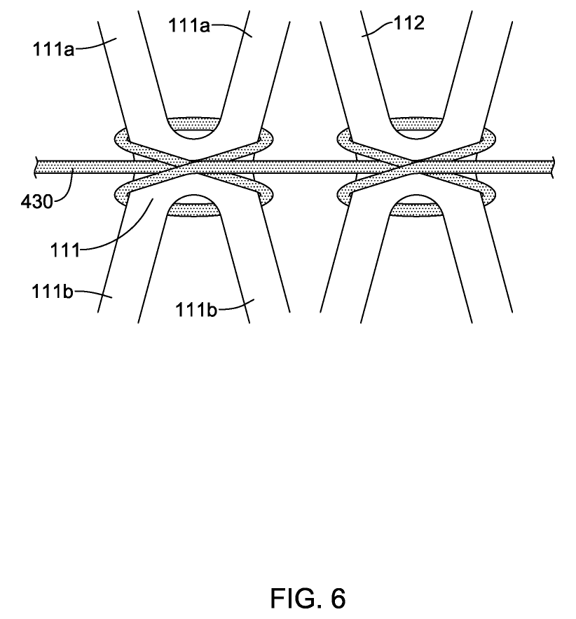

In other embodiments, a fabric mesh 430 may extend in a figure-eight around the first and second upper arms 111a and the first and second lower arms 111b of each strut junction 111. See FIG. 6. The fabric mesh 430 may then extend circumferentially between adjacent strut junctions 111, either between the stent frame 112 and the inner skirt 120 or between the stent frame 112 and the outer skirt 122.

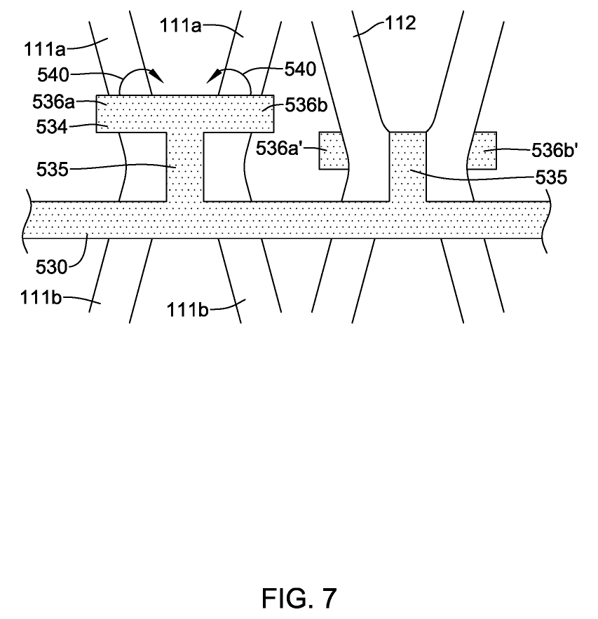

FIG. 7 illustrates a further embodiment, in which a portion of a fabric mesh 530 may be a single continuous piece that extends circumferentially around the outer surface of the stent frame 112. The fabric mesh 530 may include a plurality of T-shaped sections each including a horizontal part 534 and a vertical part 535. Each T-shaped section may be positioned such that the vertical part 535 of each T-shaped section is positioned over one of the X-shaped strut junctions 111. The horizontal part 534 of each T-shaped section may define first and second lateral arms 536a, 536b each wrapping around one of the first and second upper arms. 111a. The fabric mesh 530 may be positioned over the outer surface of the stent frame 112, under the outer skirt (not shown) with the vertical part 535 disposed over the outer surface of the strut junction 111, and the first and second lateral arms 536a, 536b wrapping from the outer surface to the inner surface between the first and second upper arms 111a, as indicated by arrows 540, and then lying flat against the inner surface of each upper arm, as shown in FIG. 7. The right side of the illustration shows the first and second lateral arms 536a’, 536b’ in their final position against the iner surface of the stent frame 112. Alternatively, the fabric mesh 530 may be positioned over the inner surface of the stent frame 112, between the stent frame 112 and the inner skirt (not shown) with the vertical part 535 disposed over the inner surface of the strut junction 111, and the first and second lateral arms 536a, 536b wrapping from the inner surface to the outer surface of each of the first and second upper arms 111a.

In any of the above embodiments, the fabric mesh 130, 230, 330, 430, 530 may have an axial height of between 1.5 millimeters (mm) and 2.5 mm. For example, the axial height may be 2.0 mm. The fabric mesh 130, 230, 330, 430, 530 may have a length of 75 mm to 90 mm. The fabric mesh 130, 230, 330, 430, 530 may have a thickness of between 30-55 micrometer (μm). For example, the thickness may be between 50 μm.

A method of manufacturing a replacement heart valve frame assembly may include providing a stent frame 112 including a plurality of struts 113 connected to one another at strut junctions 111, attaching valve leaflets 18 to the stent frame 112, wrapping a fabric mesh 130 around the stent frame 112, positioning the fabric mesh 130 to reduce leak channels around strut junctions of the stent frame 112, attaching an inner skirt 120 to an inner surface of the stent frame 112 with the fabric mesh 130 positioned between the inner skirt 120 and the stent frame 112, and attaching an outer skirt 122 to an outer surface of the stent frame 112. The fabric mesh 130 may be a single continuous piece, with a height of 1.5-2.5 mm, and a thickness between 30-55 μm. In some embodiments, wrapping the fabric mesh 230 around the stent frame 112 may include forming folds and/or pleats 236 in the fabric mesh 230 to fill and follow a profile of the stent frame 112 in leak channel areas such as the strut junctions 111, as shown in FIG. 4.

In some embodiments, the step of attaching the inner skirt 120 and the outer skirt 122 to the stent frame 112 may include stitching the inner skirt 120, fabric mesh 130, and outer skirt 122 together to the frame with a stitch pattern that causes the fabric mesh 130 to tighten circumferentially around the stent frame 112. The stitch pattern may include two rows of stitches 127 at top and bottom edges of the fabric mesh 130. In some embodiments, the stitches may extend under the stent frame 112 and then across the strut junctions 111 to form a sandwich with the inner and outer skirts. In other embodiments, the fabric mesh 330 may be wrapped around the stent frame 112 and at the strut junctions 111, the fabric mesh may be wrapped in a U shape across the top of each strut junction 111, as shown in FIG. 5. In further embodiments, the step of wrapping the fabric mesh 530 may be performed by forming the fabric mesh into a T shape at each strut junction 111, where a vertical part 535 of the T covers a front of the stent frame at the strut junction 111, and a horizontal part 534 of the T is folded down to cover an inner part of the strut junction 111 between the stent frame 112 and the inner skirt, as shown in FIG. 7.

The materials that can be used for the various components of the replacement heart valve assembly, and the various elements thereof disclosed herein may include those commonly associated with medical devices. For simplicity purposes, the following discussion refers to the assembly. However, this is not intended to limit the devices and methods described herein, as the discussion may be applied to other elements, members, components, or devices disclosed herein, such as, but not limited to, the stent frame, the inner skirt, the outer skirt, the plurality of leaflets, the fabric mesh, and/or elements or components thereof.

The inner skirt 120 and the outer skirt 122 may include a polymer, such as a thermoplastic polymer. In some embodiments, the inner skirt 120 and the outer skirt 122 may include at least 50 percent by weight of a polymer. In some embodiments, the polymer may be a polyurethane. In some embodiments, the inner skirt 120 and/or the outer skirt 122 may be substantially impervious to fluid. In some embodiments, the inner skirt 120 and/or the outer skirt 122 may be formed from a thin tissue (e.g., bovine or porcine pericardial tissue, etc.). In some embodiments, the inner skirt 120 and/or the outer skirt 122 may be formed from a coated fabric material. In some embodiments, the inner skirt 120 and/or the outer skirt 122 may be formed from a nonporous and/or impermeable fabric material. Other configurations are also contemplated.

In some embodiments, the additional system and/or components thereof may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyisobutylene (PIB), polyisobutylene polyurethane (PIBU), polyurethane silicone copolymers (for example, Elast-Eon® from AorTech Biomaterials or ChronoSil® from AdvanSource Biomaterials), ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof, may be made of a material that does not substantially distort the image and create substantial artifacts (i.e., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure, including the fabric mesh 130. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein, such as the fabric mesh 130, may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

It should be understood that this disclosure is, in many respects, only illustrative. Changes may be made in detail, particularly in matters of shape, size, and arrangement of steps without exceeding the scope of the disclosure. This may include, to the extent that it is appropriate, the use of any of the features of one example embodiment being used in other embodiments. The disclosure’s scope is, of course, defined in the language in which the appended claims are expressed.

What is claimed is:

1. A replacement heart valve assembly, comprising:
a stent frame including a plurality of struts defining a lattice structure with a plurality of strut junctions;
an inner skirt coupled to an inner surface of the stent frame;
an outer skirt coupled to an outer surface of the stent frame; and
a fabric mesh wrapped around a portion of the stent frame, wherein the fabric mesh is positioned to reduce leak channels around the plurality of strut junctions.

2. The replacement heart valve assembly of claim 1, wherein the fabric mesh has a height of between 1.5 mm and 2.5 mm.

3. The replacement heart valve assembly of claim 1, wherein the fabric mesh has a thickness between 30-50 um.

4. The replacement heart valve assembly of claim 1, wherein the fabric mesh is positioned between the inner skirt and the stent frame.

5. The replacement heart valve assembly of claim 1, wherein the fabric mesh is configured to embed into the inner skirt and fold around the plurality of strut junctions.

6. The replacement heart valve assembly of claim 1, wherein the fabric mesh comprises folds and pleats formed to fill and follow a profile of the stent frame in leak channel areas.

7. The replacement heart valve assembly of claim 1, wherein the fabric mesh is configured to promote trapping of red blood cells in small porous areas of the fabric mesh.

8. The replacement heart valve assembly of claim 1, wherein the fabric mesh includes a first layer extending circumferentially around the inner surface of the stent frame, covering a row of X-shaped strut junctions and positioned between the inner skirt and the stent frame.

9. The replacement heart valve assembly of claim 8, further comprising a second layer of fabric mesh extending circumferentially around the outer surface of the stent frame, covering the row of X-shaped strut junctions and positioned between the stent frame and the outer skirt.

10. The replacement heart valve assembly of claim 8, wherein the inner skirt and the outer skirt are attached to the fabric mesh with stitches extending through the inner skirt, the fabric mesh, and the outer skirt and around the stent frame at the X-shaped strut junctions.

11. The replacement heart valve assembly of claim 10, wherein the stitches are arranged in two circumferential rows spaced apart axially.

12. The replacement heart valve assembly of claim 1, wherein the fabric mesh is made of a material selected from polyethylene terephthalate (PET), polyamide (PA), polypropylene (PP) and polyetheretherketone (PEEK).

13. The replacement heart valve assembly of claim 1, wherein the stent frame defines a plurality of circumferentially adjacent X-shaped strut junctions that each includes first and second upper arms and first and second lower arms, wherein the fabric mesh extends between the plurality of circumferentially adjacent X-shaped strut junctions and at each X-shaped strut junction, the fabric mesh extends in an inverted U-shape over the first and second upper arms.

14. The replacement heart valve assembly of claim 1, wherein the stent frame defines a plurality of circumferentially adjacent X-shaped strut junctions that each includes first and second upper arms and first and second lower arms, wherein the fabric mesh extends in a figure-eight around the first and second upper arms and the first and second lower arms.

15. The replacement heart valve assembly of claim 1, wherein the stent frame defines a plurality of circumferentially adjacent X-shaped strut junctions that each includes first and second upper arms and first and second lower arms, wherein a portion of the fabric mesh extends around the outer surface of the stent frame, the fabric mesh including a plurality of T-shaped sections positioned such that a vertical bar of each T-shaped section is positioned over one of the X-shaped strut junctions, and a horizontal part of each T-shaped section defines first and second arms each wrapping around one of the first and second upper arms of the X-shaped strut junction.

16. A replacement heart valve assembly, comprising:
a stent frame including a plurality of struts joined at a plurality of strut junctions;
an inner skirt coupled to an inner surface of the stent frame;
an outer skirt coupled to an outer surface of the stent frame; and
at least one piece of fabric mesh wrapped around at least the plurality of strut junctions;
wherein the at least one piece of fabric mesh is positioned between the inner skirt and the stent frame, between the outer skirt and the stent frame, or between the stent frame and both of the inner skirt and the outer skirt.

17. A method of making a replacement heart valve assembly, comprising:
providing a stent frame including a plurality of stents connected to one another at strut junctions;
attaching valve leaflets to the stent frame;
wrapping a fabric mesh around the stent frame, positioning the fabric mesh to reduce leak channels around the strut junctions of the stent frame;
attaching an inner skirt to an inner surface of the stent frame with the fabric mesh positioned between the inner skirt and the stent frame; and
attaching an outer skirt to an outer surface of the stent frame.

18. The method of claim 17, wherein wrapping the fabric mesh around the stent frame includes forming folds and pleats in the fabric mesh to fill and follow a profile of the stent frame in leak channel areas.

19. The method of claim 17, wherein the fabric mesh is wrapped around the stent frame and at the strut junctions the fabric mesh is wrapped in an inverted U shape across the top of each strut junction.

20. The method of claim 17, further comprising forming the fabric mesh into a T shape at each strut junction, wherein a vertical part of the T shape covers a front of the stent frame at the strut junction, and a horizontal part of the T shape is folded down to cover an inner part of the strut junction between the stent frame and the inner skirt.
